(19) United States
(12) Patent Application Publication
(10) Pub. No.: US 2022/0254445 A1
(43) Pub. Date: Aug. 11, 2022

(54) COMPUTER IMPLEMENTED METHOD OF PERSONAL IDENTITY VERIFICATION INCLUDING BLOCKCHAIN ENHANCEMENTS

(71) Applicant: Grant A. Bitter, Agoura, CA (US)

(72) Inventor: Grant A. Bitter, Agoura, CA (US)

(21) Appl. No.: 17/695,558

(22) Filed: Mar. 15, 2022

Related U.S. Application Data
(63) Continuation-in-part of application No. 15/916,052, filed on Mar. 8, 2018.
(60) Provisional application No. 62/468,532, filed on Mar. 8, 2017.

(51) Int. Cl. G16B 20/20; H04L 67/12; H04L 9/40

(52) U.S. Cl. CPC G16B 20/20; H04L 63/102; H04L 63/0861; H04L 67/12

(57) ABSTRACT

This invention discloses an automated computer method for personal identity verification with improvements over existing technology. Specific embodiments of the invention ensure that an individual's personal identity data may be electronically maintained in databases and used for identity verification with a high level of security. The invention greatly minimizes the possibility of identity theft and identity fraud compared to existing methodologies. The process employs a plurality of networked computers and application programming interfaces. In certain embodiments the process is used in multifactor authorization to login to a server, network or online accounts (e.g. private databases, internet websites). Additional features that improve the basic identity verification process are provided through incorporation of blockchain technology. The computer implemented process is initiated by receiving an electronic request, from an entity representing to be a certain individual, that requires verification to proceed. Various embodiments of the invention utilize previously determined sequence information for the given individual stored in a separate secure database to either confirm or deny the electronic request. As part of this process, certain data is required to be electronically submitted by the requesting entity as credentials. Various computational methods are then used to determine whether the data credentials electronically submitted correspond to the sequence information of the given individual stored in the database. If the computations yield concordance between the electronically submitted data credentials and the given individual's data in the database, the computer system allows the electronic request to proceed; if the computation detects discrepancy between the two datasets the electronic request is rejected.

### CROSS REFERENCE TO RELATED APPLICATIONS

This application is a continuation-in-part and claims priority to U.S. non-provisional application Ser. No. 15/916,052 filed on Mar. 8, 2018, which claims priority to U.S. provisional Application No. 62/468,532 filed on Mar. 8, 2017; each of which are hereby incorporated by reference in their entirety.

### BACKGROUND OF THE INVENTION

Personal identity verification is necessary for a wide spectrum of security purposes; indeed it is performed pervasively in modern society. These activities range from logging into a personal computer to applying for credit cards or loans. Financial institutions typically use static identifying information such as name, date of birth, driver license number and social security number; in other instances personal passwords, etc. are used. Security breaches in which such personal identifying information is stolen (identity theft) from computer networks and databases have become common. In each of several different cases, the data of nearly 50% of the U.S. population was obtained. It is not unreasonable to assume that the personal identifying information of a large majority of Americans has been compromised. These data could potentially be used for identity fraud at any time in the future. Financial losses due to identity fraud have historically increased, and dramatically so in recent years. In addition to direct financial losses, identity fraud results in numerous other security and societal problems. There is a need for improved, more secure and robust methods of verifying personal identity.

### SUMMARY OF THE INVENTION

This invention discloses a computer implemented method for verification of an individual's identity by computational analysis of genomic DNA. The method provides an identity verification that is orders of magnitude more precise and more secure than previous or current methods. The method involves utilizing DNA sequence information from the individual's genome to either confirm or deny a request for identity verification. The identity verification process is initiated by submitting a DNA sequence and requesting identity verification as said individual. The authentic genome of the individual for whom identity verification is sought is then interrogated by various computational methods to generate a determined DNA sequence for the genomic positions submitted for identity verification. If there is concordance between the submitted DNA sequence information and the determined DNA sequence, the request for identity verification is confirmed. If there are difference(s) between the submitted DNA sequence information and the determined DNA sequence, the identity verification request is denied.

### ABBREVIATIONS

“Admin”, Administrator of the DB; “API”, application programming interface; “bp”, base pair; “DB”, Database; “DNA.geno”, genomic DNA sequence information of IP that is used in IDV; “DNA.pos”, DNA nucleotide positions in a human genome; “DNA.seq”, DNA nucleotides present at the positions specified in DNA.pos; “DNA.test”, the result obtained when the nucleotides present at DNA.pos are extracted from a DNA.geno; “IDV”, Identity Verification Process; “IP”, Individual Person; “OL”, Other Laboratory; “SNP”, Single Nucleotide Polymorphism.

### BRIEF DESCRIPTION OF THE DRAWINGS

FIG. 1. Process diagram of identity verification by computational analysis of genomic DNA.

FIG. 2. Process diagram of identity verification for an individual person, and denial of identity verification for a fraudulent request, by computational analysis of genomic DNA.

FIG. 3. Process diagram of identity verification for an individual person, and denial of identity verification for a fraudulent request, by computational analysis of genomic DNA that is processed through a third party service.

FIG. 4. Process diagram of identity verification for an individual person, and denial of identity verification for a fraudulent request, by computational analysis of genomic DNA that is processed through a third party service in response to data submitted to a Financial Institution.

FIG. 5. Process diagram of prior art of identity verification for an individual person, and denial of identity verification for a fraudulent request, that is performed by a third party service in response to data submitted to a Financial Institution.

FIG. 6. Process diagram of identity verification for an individual person by computational analysis of genomic DNA that uses a digital signature to identify said individual person.

FIG. 7. Comparison of the architecture of centralized server based networks and that of a blockchain peer to peer network.

FIG. 8. Process diagram for logging into an online account and using identity verification by computational analysis of genomic DNA for multifactor authorization.

### DETAILED DESCRIPTION OF THE INVENTION

This invention provides a highly specific method for verification of an individual's identity by computational analysis of genomic DNA. The computational methods and technology procedures disclosed for the identity verification process (IDV) represent significant improvements over other existing identity verification methods.

The invention utilizes genomic information of an individual person (IP) to either confirm or not confirm the identity of a requesting entity as the IP.

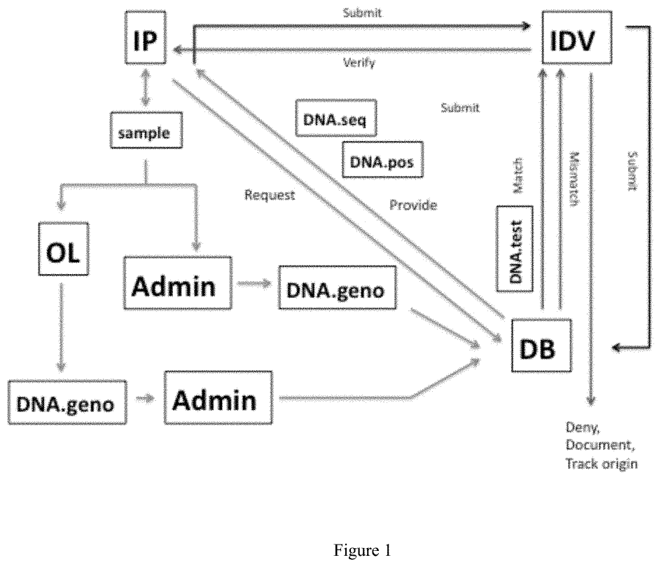

As depicted in FIG. 1, the IDV requires genomic DNA sequence information of the IP. This information can be determined as a first step in the IDV. In one embodiment of the invention a sample, such as cheek cells collected with buccal swabs or saliva, is obtained from the IP and submitted to the Administrator of the DB (Admin). Genomic DNA is extracted, and the DNA nucleotide sequence information is determined by any of a wide variety of methods that are available. These methods include, but are not limited to, next generation sequencing (NGS), dideoxy chain termination DNA sequencing (Sanger sequencing), chemical degradation DNA sequencing (Maxam-Gilbert sequencing), nanopore DNA sequencing, SNP detection using microarrays, allele-specific PCR amplification, quantitative PCR amplification, etc. These methods of obtaining DNA sequence information of IP are referred to collectively as “sequencing”, and the data obtained referred to variously as “DNA sequence”, “sequence”, “genetic information”, “genotype” etc. of IP. The DNA sequence so determined (DNA.geno) may be stored in a variety of file formats including, but not limited to, *.fasta, *.bam, *.abl, *.seq, *.gb, *.txt, *.csv, and *.vcf files. This DNA.geno is associated with and linked to the IP, and is used to verify the identity of IP.

The DNA.geno obtained by this analysis can be stored in such a way that it can be used in subsequent IDV. Referring to FIG. 1 as an example, the DNA.geno may be stored in a database (DB) which is a computer or server that can have any one of a number of available operating systems. In a preferred embodiment, the Unix operating system is used and computational procedures may be performed with Unix commands, or any one of a number of available software programs such as Python. In a preferred embodiment, the DB allows receiving and sending of files via the internet, and may be encrypted and may also have multiple other layers of security to prevent unauthorized access to information in the DB.

In another embodiment of the invention, the DNA.geno of IP may have been previously determined by other entities using a variety of methods that are available. Each such entity is referred to as other laboratory (OL). In this embodiment, the DNA.geno of IP determined by OL is deposited into the DB and is uniquely associated with and linked to IP. Depositing the DNA.geno into the DB can be done using a variety of available file transfer protocols. In a preferred embodiment, as depicted in FIG. 1, the DNA.geno is submitted to the same Admin that administers the embodiment above, wherein a sample from IP is submitted to Admin for DNA sequence analysis and subsequent deposit of DNA.geno into the DB.

The IDV employs the following general steps as further illustrated in FIG. 1. An individual wishes to represent to an entity that they are, in fact, IP. The entity may be, for example but not limited to, any individual, organization, business or government agency. The DNA.geno of IP was previously deposited in the DB. The individual seeking the identity verification submits two files to the IDV. IDV refers to the identity verification process, and may involve any one or more entities including Admin, third party services and identity verification customers. One file lists specific nucleotide positions in a human genome (DNA.pos). In one embodiment, DNA.pos lists the positions (or coordinates) according to the nomenclature of a reference human genome assembly (https://www.ncbi.nlm.nih.gov/grc/human). These are typically represented by chromosome number and nucleotide position on one strand (plus or minus) of that chromosome. For whole chromosomes, or subsets thereof, the DNA sequence can be represented by chromosome number, starting nucleotide and ending nucleotide on a specified strand of the reference human genome assembly. In another embodiment, DNA.pos may refer to specific single nucleotide polymorphisms (SNPs) in the human genome which are identified by rsID numbers. Examples of this nomenclature appear below. The second file submitted to the IDV provides the actual nucleotides (DNA.seq) that occur at those positions in IP's DNA.geno. In a preferred embodiment, as depicted in FIG. 1, the DNA.pos and DNA.seq files are provided to IP by DB in response to a request initiated by IP After submission of an identity verification request to IDV (by providing DNA.pos and DNA.seq files), IDV submits both files received to the DB and requests verification by DB of the submitting entity as IP. From the DNA.geno of IP deposited in the DB, visual inspection or various computational methods that are available are then used to extract the nucleotides present at DNA.pos, and the data is outputted to a file named DNA.test. The DB then uses visual inspection or available computational methods to compare DNA.seq and DNA.test, and if there is perfect concordance between the two files confirms to IDV the identity as IP. If there are any differences between DNA.seq and DNA.test, verification of the entity submitting the files as IP is denied.

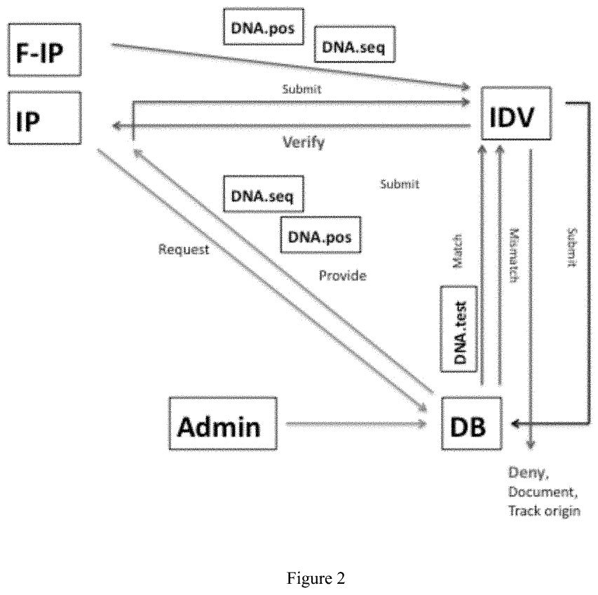

Consider two possible scenarios in the above IDV. Referring to FIG. 2, the first individual seeking the identity verification is an IP and has access to their DNA.geno in the DB. IP submits a request to the DB to initiate IDV. DB then provides two files, DNA.pos and DNA.seq, which IP submits to the IDV. This DNA.pos file includes all or a subset of the nucleotide positions that were determined by Admin or OL during sequencing of IP's genome and deposited as file DNA.geno into the DB. The DNA.seq file lists the actual nucleotides present in IP's DNA.geno that are present at each position in DNA.pos. Upon submission to IDV, DNA.pos and DNA.seq are then submitted back to the DB. From the DNA.geno of IP deposited in the DB, manual or any one of a number of computational methods that are available is used to extract the nucleotides present at DNA.pos, and the data is outputted to a file named DNA.test. The DB then uses visual inspection or available computational methods to compare DNA.seq and DNA.test. In this case, since the DNA.seq was originally extracted from IP's DNA.geno, and the computational analysis is also performed on IP's DNA.geno, there is perfect concordance between the two files. The DB then confirms the submitted data as corresponding to IP's DNA.geno. The identity of the submitting entity as IP can be confirmed as the last step in the IDV.

In contrast, the second entity seeking identity verification as IP is not IP and is designated F-IP in FIG. 2. F-IP does not have access to IP's DNA.geno, but still needs to submit the two types of files required to initiate the IDV. Upon submission to IDV, DNA.pos and DNA.seq are then provided to the DB. In one embodiment, the IDV may be immediately denied if F-IP submits a DNA.pos that was not determined or only partially determined in IP's DNA.geno. Since F-IP does not have access to IP's DNA.geno, F-IP must necessarily guess IP's DNA.seq, or repeatedly try different DNA.seq submissions, to obtain verification as IP. The probability of correctly identifying IP's DNA.seq decreases as the amount of DNA sequence information included in DNA.pos increases. This is discussed further below and, by including enough genomic DNA coordinates in DNA.pos, the probability of guessing or randomly selecting IP's DNA.seq for that DNA.pos becomes vanishingly low. From the DNA.geno of IP deposited in the DB, manual or any one of a number of computational methods available is used to extract the nucleotides present at DNA.pos, and the data is outputted to a file named DNA.test. The DB then compares DNA.seq and DNA.test. Without knowledge of IP's actual DNA.geno, the DNA.seq submitted by F-IP will almost certainly be different than DNA.test. In this case, verification of F-IP as IP is denied.

For simplicity of presentation, the process diagrams of FIGS. 1 and 2 depict the IDV in terms of information flow between IP and DB. As indicated in the figures, the specifics of this information flow are designed, maintained and administered by Admin, and it can be automated using available technologies. It is appreciated that, in various embodiments of the invention, additional entities or technologies, which are available to one of ordinary skill in the art, will be employed in this process of information flow between IP and DB.

In one embodiment, the IDV utilizes file transfers between computers and servers via the internet, and either verifies or denies verification of a submitting entity as IP (FIGS. 1-2). In an additional embodiment of the invention, for the case of a denial of identity verification, the technology of this invention can be used to track the origin of the fraudulent IDV request. This may be done, for example, by tracking IP addresses during the course of submitting and processing the IDV request. In other embodiments, the invention may be practiced using computers and servers that interact using non-internet based communication.

In a preferred embodiment of the invention, the DNA.pos and DNA.seq provided by DB in response to IP's request is transmitted to the IDV in an automated process. Software associated with IP and utilized to request the data automatically submits it to IP's designated IDV. This would eliminate the potential for human (IP) error in submission of the data to IDV.

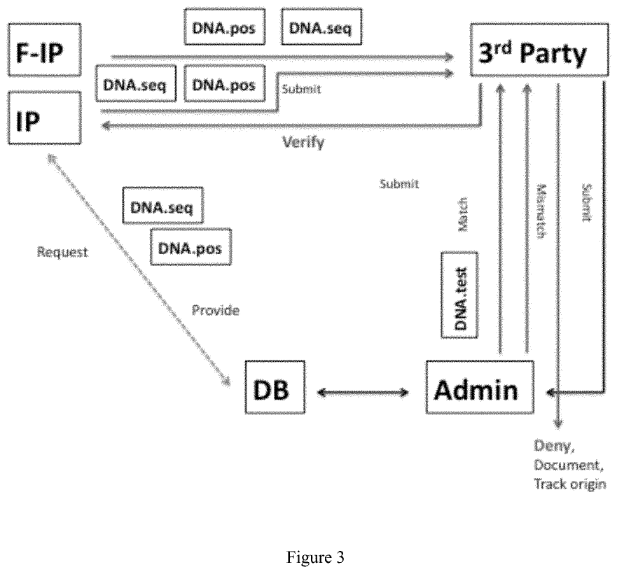

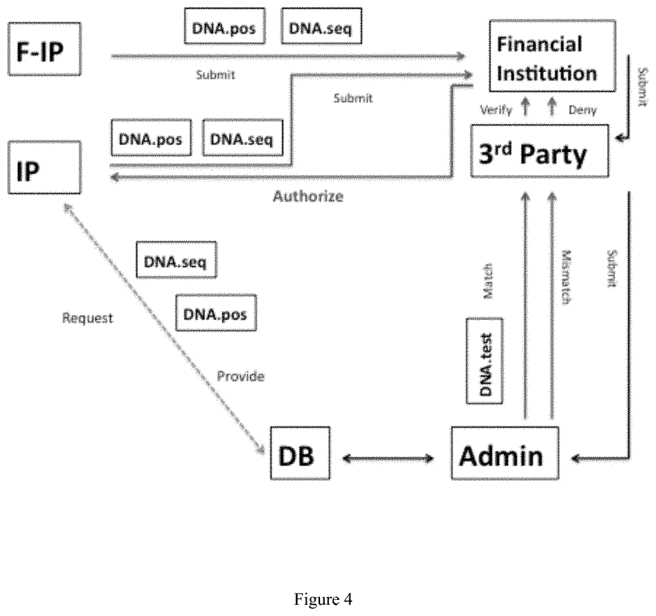

It is appreciated that, in one embodiment of the invention, the identity verification process (DV) can be completely conducted by the Admin that administers the DB. In this embodiment, IDV of FIGS. 1 and 2 is Admin. In another embodiment of the invention, IDV may be a third party (or several parties). The third party may be, for example, an identity verification service that contracts with various entities to verify the identity of individuals interacting with said entities. This embodiment is indicated schematically in FIG. 3. One example of such a third party is an identity verification service that provides background checks to financial institutions, such as credit card companies or banks, to confirm the identity of an IP applying for a credit card or loan. This mode of IDV is depicted in FIG. 4. In this embodiment, individuals apply to a financial institution to obtain a credit card in the name and identity of IP. The financial institution requests proof that the requesting entity is IP and, according to the invention, this consists of DNA.pos and DNA.seq files. The financial institution then provides this data to the third party that then forwards it to the Admin of the DB. The IDV is performed as described above, and Admin provides the result (match or mismatch) to the third party which then relays this to the financial institution. The financial institution then authorizes the credit to IP, or denies (does not authorize) the credit application, based on the IDV result.

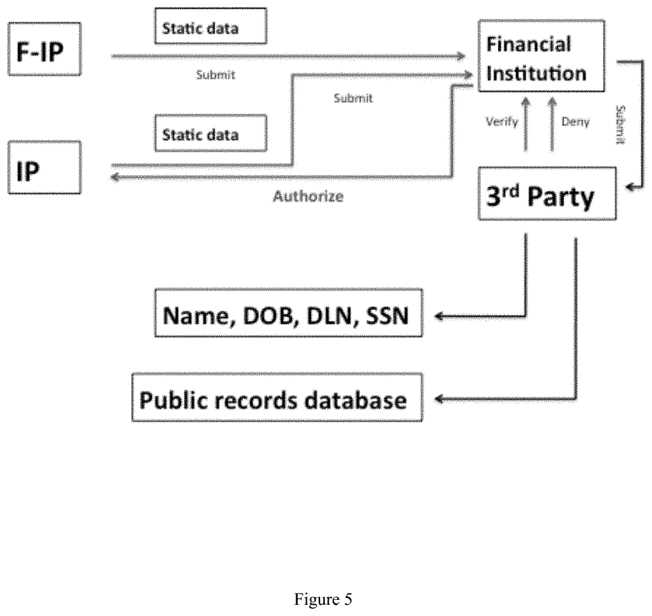

The embodiment of the invention for IDV to obtain credit or a loan as depicted in FIG. 4 can be contrasted with the methods in use at this time. The prior art is schematized in the process diagram of FIG. 5. The financial institution requests certain information from individuals applying for credit or a loan in order to verify their identity. The initial information requested is typically name, date of birth and social security number. Financial institutions generally contract with a third party identity verification service (“3^{rd} Party”) (https://en.wikipedia.org/wiki/Identity verification service) to confirm that information. The 3^{rd} Party also searches public, and sometimes private, databases for additional information on IP. This includes data such as drivers license, current and previous addresses and phone numbers, current and former employers, closest relatives, etc. From this analysis, an “identity score” is generated and certain criteria used to either verify or deny the identity as IP. In contrast to the invention (FIG. 4), the data collected with current methods is quite limited (FIG. 5). Furthermore, that data is static and if obtained by others could be fraudulently used by an F-IP to obtain identity verification as IP. With prior art methods and procedures, there have been numerous instances of stolen identity and resulting fraud. Identity theft fraud has been increasing, and it is estimated that it resulted in over $16 billion in losses directly to consumers in 2016.

This invention introduces new attributes to the process of identity verification:

1. The data is an actual biologic property of the IP.
2. Each individual has a unique genome, and their genome DNA sequence distinguishes them from others.
3. A large magnitude of genomic DNA sequence data for each IP is available to use in IDV.
4. The data used for IDV can be generated dynamically.
5. Because of the magnitude of DNA sequence variations between individuals, it is possible to use subsets of genomic DNA in IDV without ever reusing the same DNA sequence information for an IP.
6. The data used for IDV can be determined and stored in strict confidence, without ever appearing in any public or other private database.

The DNA sequence information determined from IP's sample and utilized in the IDV (DNA.geno) may be any portion, portions or all of the DNA sequence in IP's full genome. It is appreciated that the entire DNA sequence of IP's genome, consisting of approximately 3 billion (3×10⁹) bp of sequence, will capture the most genetic information and variation that is present in each IP, and could be deposited in the DB. Nevertheless, the invention can be practiced by depositing a subset of the DNA sequence from IP's genome into the database. This subset can be of numerous types, and various combinations thereof. At the minimum, it could be one nucleotide of IP's entire genomic DNA sequence although this would severely limit the security, and thus utility, of the invention. A wide variety of subsets of IP's entire genomic DNA sequence could be deposited in the DB. These include:

the DNA nucleotide sequence (either or both strands) of the entire genome of IP,

the DNA nucleotide sequence of any subset of the entire genomic DNA sequence of IP,

any combination of DNA nucleotide sequence subsets, either overlapping or non-overlapping, of the entire genomic DNA sequence of IP,

any DNA sequence information that includes all or portions of regions where insertions, deletions, inversions and/or repeats of DNA occur in the genome of IP,

any DNA nucleotide sequence information that includes, or is, specific non-contiguous nucleotides from IP's genome. These may, for example, correspond to single nucleotide polymorphisms (SNPs) in IP's genome. For each genomic position, the specific nucleotide (G, A, T or C) on one or both strands may be included in the DNA.geno file. In the case of SNPs, since most human cells are diploid, the IP's genotype may be heterozygous. For example, one allele may have a G whereas the other allele may be A at that position on a given strand (plus or minus) in the genome. This heterozygous SNP genotype is reported with the generic nomenclature rsID GA, as depicted in the specific examples below.

The draft version of the human genome sequence was published in 2001, and the finished version of the human genome sequence completed in 2003. That DNA sequence was determined on genomic DNA from a small number of individuals. All individuals have unique genomes, and the term “human genome sequence” as it is commonly used is more accurately stated as a human “reference” genome sequence. In the years since 2003, DNA sequencing technologies have improved, many more individual human genomes have been sequenced, and the human reference genome DNA sequence has been refined. Notably, in the genome sequence completed in 2003 there were “gaps” in the DNA sequence. These were primarily regions of chromosomal DNA that were difficult to sequence due to structural or specific DNA sequence issues. As the human genome DNA sequence is refined, sequential reference human genome assemblies are published (https://www.ncbi.nlm.nih.gov/grc/human). The positions (or coordinates) of specific nucleotides may vary between reference assemblies. For example, below are depicted SNP genotype results from one individual as they appear in two different human reference genome assemblies. Four homozygous and one heterozygous (rs11240777) SNPs appear in this example.

Reference Human Assembly Build 36

| rsID | Chromosome | Position | Genotype |
|---|---|---|---|
| rs4477212 | 1 | 72017 | AA |
| rs3094315 | 1 | 742429 | AA |
| rs3131972 | 1 | 742584 | GG |
| rs12124819 | 1 | 766409 | AA |
| rs11240777 | 1 | 788822 | AG |

Reference Human Assembly Build 37

| rsID | Chromosome | Position | Genotype |
|---|---|---|---|
| rs4477212 | 1 | 82154 | AA |
| rs3094315 | 1 | 752566 | AA |
| rs3131972 | 1 | 752721 | GG |
| rs12124819 | 1 | 776546 | AA |
| rs11240777 | 1 | 798959 | AG |

For each SNP, the chromosome number and genotype remain the same in each genome assembly build. However, the position on chromosome 1 of each SNP in assembly build 37 is different from assembly build 36. This is apparently due to the presence of an additional 10,137 bp of DNA sequence on chromosome 1 before the position of rs4477212 in assembly build 37.

In a preferred embodiment of the invention, for DNA.geno deposited in DB a notation of the human genome reference assembly to which the nucleotide coordinates refer will be associated with the file. Additionally, if the DNA.geno is single stranded, the strand in the human genome reference assembly (e.g. plus or minus) to which it corresponds will be noted.

It is appreciated that, in other embodiments of the invention, the IDV can be performed using other samples and macromolecules from IP. Other samples are for example, but not limited to, various biologicals from IP such as blood, hair, skin or various tissues. Other macromolecules include RNA and protein, and both are indirect measurements of genomic DNA sequence. Each type of macromolecule can be subjected to sequence analysis for the purpose of IDV. However, DNA sequence analysis is currently much faster and less expensive, and is the preferred embodiment of the invention. Other types of cellular (extra-chromosomal) DNA from IP, such as mitochondrial DNA, could be used in IDV but the magnitude of information content is much less than that contained in genomic DNA.

Most currently used identity verification methods utilize static data. Examples of static data are date of birth, social security number, mother maiden name, fingerprints, eye scans, retina scans, etc. Static data remains constant and does not change. If an IP's static data is obtained, it could be used by someone else to fraudulently obtain verification as IP. Current methods for identity verification also employ “semi-static” data such as name of favorite pet, best friend, favorite sport, etc. This “semi-static” data may be inputted by IP and can be changed, but is used repeatedly until changed by IP.

One advantage of this invention is the magnitude of data available for IDV, and the fact that it does not need to be static. The DNA.pos used in the IDV may be all or any subset of DNA.geno. The DNA.pos used in the IDV may be the same for each subsequent IDV. This would be necessary in the case where the entire DNA.geno is used as DNA.pos. In other embodiments of the invention, a subset of the DNA sequence information present in DNA.geno may be used as DNA.pos. Utilization of a subset of DNA.geno allows submission of different DNA.pos in subsequent IDV for IP. This embodiment of the invention can introduce substantial improvement in security of IDV. By utilizing different DNA.pos in subsequent IDV, the potential security/confidentiality breach that could occur if the data in an IDV transaction were somehow intercepted or misappropriated by an unauthorized party can be minimized or eliminated. That is, if an unauthorized party obtains the IDV data of IP (DNA.pos, DNA.seq and/or DNA.test), its use in future IDV for IP can be rejected. In this embodiment of the invention, the DB retains the DNA.pos that was used for each IDV of IP, and issues a new DNA.pos for each subsequent IDV. In the preferred embodiment these new DNA.pos are unique, but they could also be partially overlapping. In the event that the same DNA.pos is used again to request IDV for that IP, the request can be denied. As discussed below, with appropriate DNA.geno and DNA.pos files, it may be possible to maintain an extremely high level of IDV security and never use the same DNA.pos more than once during an IP's lifetime.

One aspect of the novelty of this invention is the amount of data that can be used for IDV. The human genome consists of approximately 3 billion (3×10⁹) bp of DNA distributed among 46 total chromosomes in diploid cells. To date, approximately 39 million (39×10⁶) single nucleotide polymorphisms (SNPs) have been identified in humans. SNPs are positions in the human genome where nucleotide differences exist between individuals. One example of a SNP is rs1815739 in the human ACTN3 gene (https://www.ncbi.nlm.nih.gov/snp/?term=rs1815739). The DNA sequence on the coding strand of the two forms of the gene are depicted below with the SNP nucleotide indicated in color.

Wild type (R allele)
5′-CTGCCCGAGGCTGAC [symbol] GAGAGCGAGGTGCCA -3′
Mutant (X allele)
5′-CTGCCCGAGGCTGAC [symbol] GAGAGCGAGGTGCCA -3′

The wild type gene has a C whereas the mutant gene has a T at this position (SNP) in the genome.

Each human, on average, has approximately 3.6 million (3.6×10⁶) SNPs in their genome. These DNA sequence variations are in addition to other types of DNA sequence alterations, such as insertions, deletions, duplications, inversions, etc. The genome of each individual is unique, with differences having been observed even between identical twins.

SNPs can be used as one example to demonstrate the magnitude of information content in genetic variation. In the ACTN3 gene example above, these are either C or T on the coding strand. For diploid cells, such as human somatic cells, there are two copies of each gene. For this SNP, therefore, there are three possible genotypes for diploid cells (using the sequence on the coding strand above): CC (homozygous wild type), CT (heterozygous) or TT (homozygous mutant).

Next, consider the case for two SNPs. In this example, the second SNP can be either A or G, and there are three possible genotypes in diploid cells: AA, AG, or GG. The possible genotypes for this combination of SNPs are:

| SNP1 | SNP2 |
|---|---|
| CC | AA |
| CC | AG |
| CC | GG |
| CT | AA |
| CT | AG |
| CT | GG |
| TT | AA |
| TT | AG |
| TT | GG |

The number of possible genotypes for genetic data consisting of the following number of SNPs, each with two possible nucleotides, is:

$$\begin{matrix}1 SNP = 3 \\ 2 SNPs = 3 \times 3 = 9 \\ 3 SNPs = 3 \times 3 \times 3 = 27 \\ ⋮ \\ n SNPS = {3}^{n} \\ e . g . {3}^{10} = 59 , 049 \\ {3}^{15} = 14 , 348 , 907 = 14 \text{.348} \times {10}^{6} \\ {3}^{20} = 3 , 486 , 784 , 401 = 3.486 \times {10}^{9} \\ {3}^{30} = 205 , 891 , 132 , 094 , 649 = 205.891 \times {10}^{12} \\ {3}^{64} = 3 , 433 , 683 , 820 , 292 , 510 , 000 , 000 , 000 , 000 , 000 = 3 \times {10}^{30} \\ ⋮ \\ {3}^{3 , 6000 , 000}\end{matrix}$$

is the approximate number of possible genotypes using all the SNPs that are present in an average individual's genome.

The number of possible genotypes using SNPs is even greater than that indicated above, since those represent only di-allelic SNPs. Some SNPs contain three possible nucleotides and others contain all four possible nucleotides (collectively referred to as k-allelic SNPs). If such SNPs are included in the genetic data, the number of possible genotypes would be accordingly larger than that indicated above.

Even with a small number of SNPs such as 64, which are commonly used in pharmacogenetic testing panels, a very large number of possible genotypes exist. 3×10³⁰ is more than 1,000,000 trillion trillion (10³⁰=10⁶×10¹²×10¹²). The probability of randomly selecting an IP's genotype from just these 64 SNPs alone is vanishingly small. Inclusion of more SNPs (of the approximately 3.6×10⁶) in IP's genome can make the odds of randomly selecting an IP's genotype “astronomically” low.

Compare these probabilities to those of other identity verification methods. One identifier of individuals in the U.S. is social security number (SSN), which has the generic format xxxx-yyy-zzzz. Since each position has one of 10 possible digits (0-9), there are 10¹¹ possible SSN. Only a small subset of the SNPs in an IP's genome (64 of 3.6×10⁶ in the example above) provide a match probability (one in 10³⁰ in the example above) which is exceptional compared to all other current identity test methods. The security of the IDV in this invention can be made even greater by including more SNPs. Furthermore, the maximum total number of non-overlapping DNA.pos of 64 SNPs is:

$$( 3.6 \times {10}^{6} ) / 64 = 56 , 250$$

If partially overlapping DNA.pos (some SNPs are shared between two DNA.pos but others are unique) are used, the number of possible DNA.pos which are non-identical (and non-recurring) increases dramatically. Utilizing various combinations of DNA sequences from different regions of IP's genome in DNA.pos would also dramatically increase the total possible number of non-identical DNA.pos.

In the preferred embodiment of this invention, two criteria in the IDV must be met in order to confirm identity. First, the DNA.pos submitted as part of the IDV must include only nucleotide positions that were determined and are contained in DNA.geno. Second, the DNA.seq submitted to IDV must correspond exactly to DNA.test. In other embodiments, DNA.pos submitted that include nucleotide positions not present in DNA.geno could be processed in the IDV. In this case, identity could be confirmed if the nucleotides of DNA.pos that are present in DNA.geno correspond to those in DNA.seq submitted to IDV. Although it could compromise the security and accuracy of the IDV, a DNA.test that is only partially (for example 98%) identical to DNA.seq could be used to confirm identity. This less desirable embodiment might be useful to accommodate certain uncertainties in experimental data (e.g DNA sequence determination) or statistical limitations of computational methods.

In one embodiment of the invention, a different DNA.pos is used in each IDV of IP. Because of the magnitude of the information content using SNPs, and the accordingly increased information content when other types of DNA sequence alterations are included, it is possible to practice this invention without ever using a given DNA.pos for IDV more than once for each IP. This aspect of the invention dramatically improves the security of this IDV over previous or existing identity verification methods.

Another advantage of the current invention over other identifiers, such as name, date of birth and SSN, is that the DNA.geno is a biologic characteristic of the IP. In the IDV depicted in FIGS. 1-4 and 6, the DNA.geno data has been experimentally determined previously from a sample collected from IP, and the data can be accessed and utilized repeatedly. DNA.geno could also be determined on IP, and/or any other entity requesting an IDV as IP, at a later date if desired or necessary. This may be appropriate in the case of suspected IDV security breach.

The IDV of the invention is highly specific, and many orders of magnitude more precise and secure than previous or current methods. By the nature of this invention, it will be very difficult for an unauthorized person (F-IP in FIGS. 2, 3 and 4) to fraudulently receive verification as IP. The IDV of the invention involves complex manipulation of genomic data and this data can be generated dynamically. Other embodiments of the invention described herein allow additional security features. In contrast, current identity verification requires a limited amount of static data of the IP. Furthermore, the expertise to practice the invention, and as a corollary to exploit it, is quite specialized and limited.

The DB that stores DNA.geno for each IP must be maintained securely to prevent a data breach. This is true for all data storage systems, and state of the art security systems should be used at all times to maintain the DB. One example is blockchain technology. https://en.wikipedia.org/wiki/Blockchain).

Although proposed earlier, the first computer application of blockchain technology was published as a whitepaper in 2008 (Satoshi Nakamoto, www.bitcoin.org “Bitcoin: A Peer-to-Peer Electronic Cash System”). In January 2009 Bitcoin was released to the public as open source software; it was freely available for anyone to use, edit or modify for other applications. Since the publication and availability of this technology, blockchain has achieved use in a number of settings and there has developed a field of knowledge [reviewed in Hellwig, D. et al. (2020) Build Your Own Blockchain—A Practical Guide to Distributed Ledger Technology © Springer Nature Switzerland AG] that is currently being explored for new applications.

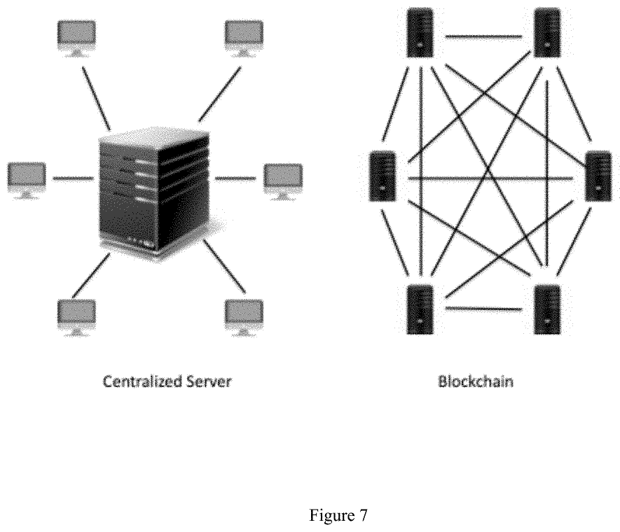

Fundamentally, a blockchain consists of a peer to peer (P2P) network of independent computers or servers (termed “nodes” of the blockchain). Typically these are at physically different locations, exist over wide geographic regions and can include hundreds or thousands of independent nodes connected in a network. The architecture of a blockchain is contrasted in FIG. 7 with that of the traditional and more common network that employs a centralized server. P2P networks are more secure and more resilient than centralized server based networks since they do not have a single point of failure. For a blockchain to be hacked and maliciously compromised by any entity would require successfully accessing more than 50% of the physically and geographically distinct nodes. The first feature that blockchain introduces to the IDV process is more secure storage of data. The IDV process of the instant invention either confirms or denies a request to be verified as a given individual based on submitted genomic DNA sequence information (DNA.pos and DNA.seq). Maintaining the IP DNA.geno used in IDV in a blockchain essentially removes the potential for identity theft that has plagued current identity verification methods.

Distributed ledger technology (DLT) refers to a data structure that resides across multiple computers or servers linked in a P2P network and spread across multiple locations. Blockchain is a subset of DLT that describes a data structure that stores a permanent history of transactions. The primary activity of a blockchain is the chronological linking of blocks of data in a chain; the first block is referred to as the genesis block, and new data is added as a subsequent block. A key feature of blockchain is timestamping; every transaction and every block includes a timestamp. The combination of cryptographic hashing with timestamping (below) renders the data in a blockchain unchangeable. Thus, the second feature that blockchain can introduce is an immutable ledger of all transactions that have occurred. It is noted that in addition to adding data, blockchain data could be changed or deleted. That would, however, require doing so in a new block that is first validated by a consensus of the blocks (below) before adding to the blockchain.

Blockchains can be either public, private, or a hybrid consortium with various types of permissions. A public blockchain can be joined by anyone from anywhere; it is termed a “permissionless” blockchain. No individual or computer is responsible and any node can be created and participate in reading, writing and verifying the blockchain. Public blockchains are open and transparent, with the Bitcoin and Ethereum cryptocurrency blockchains currently being the most prominent. In contrast, private blockchains require pre-verification of any participating node in the blockchain. Since there is a central point of control verifying new nodes, private blockchains may be more susceptible to failure. Accordingly the nodes in such blockchains typically are run by parties that know (and trust) each other. Private entities that are particularly concerned with data privacy and control employ private blockchains. Finally, consortium-controlled blockchains are an extension of private blockchain technology, but they do not employ a central authority that verifies new nodes. Rather, a consortium could consist of a fixed number (e.g. 30) of entities and specify that any decision or transaction is accepted as valid only if more than 50% (16 or more for this example) of the entities confirm it.

New data is added as a block to the blockchain only after it has been validated by a consensus of the network nodes. Consistency of a blockchain refers to an agreement amongst the nodes that the stored data is an accurate representation of all changes that have been made since the genesis block and the sequence of these events; this is used in DLT to ensure that all nodes have an identical copy of the distributed database. There are a variety of consensus protocols that are employed, including
Proof of Work (PoW)
Proof of Stake (PoS)
Proof of Capacity/Proof of Space
Delegated Proof of Stake (DPoS)
Proof of Authority (PoA)
Practical Byzantine Fault Tolerance (PBFT)
Proof of Elapsed Time (PoET)
   and others. Blockchain technology could be integrated in the IDV process of the instant invention in numerous variations.

As discussed above, the magnitude of human genomic DNA sequence information allows maintaining a high level of security using a small subset of the total genomic information. Therefore, it is possible to use multiple DNA.geno of IP (e.g. DNA.geno₁, DNA.geno₂, DNA.geno₃, etc.). To improve security, use of these different DNA.geno for each IP stored in the DB could be periodically changed for IDV.

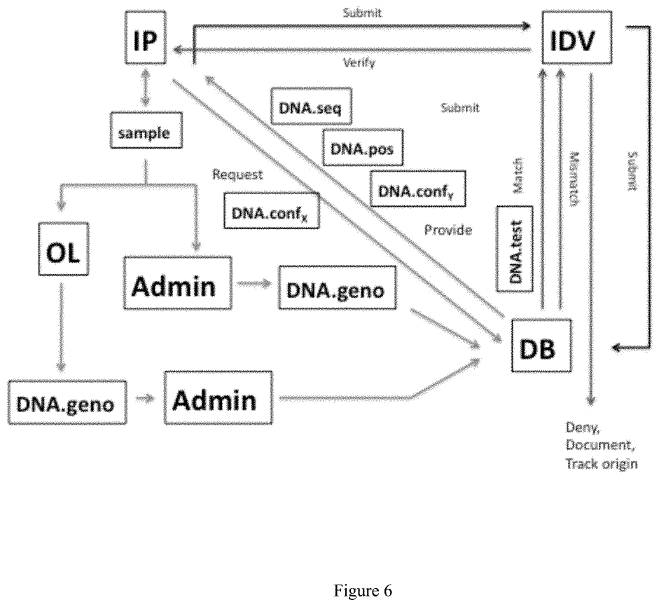

In yet another embodiment of the invention, a digital signature may be used to track and confirm the entity that submits the IDV request. As one example of this embodiment, when the IP's account is created and DNA.geno is deposited, a DNA.seq may be extracted from IP's DNA.geno. This DNA.seq is termed DNA.conf and returned to IP and also associated with IP's account in the DB. For each IDV made by or on behalf of IP, the DNA.conf can also be required by DB to authenticate the requesting entity before processing the IDV. To further improve security, periodically or for each IDV processed and verified, a new DNA.conf can be issued to IP. This could be, for example and by way of convenience, the DNA.test that is generated and does confirm the identity of IP during an IDV. This example of a digital signature may similarly be changed in each subsequent IDV. The then existing DNA.conf for IP can be required for each new IDV as an added security that only requests from the bona fide IP will be processed. Two aspects of this embodiment contribute to the additional security of IDV. First, the DNA.conf does correspond to IP's DNA.geno information. Second, the DNA.conf is changed after each IDV transaction. This embodiment of the invention is depicted in FIG. 6.

There are a number of additional embodiments of the invention that can improve the robustness and security of the IDV. Among these are the use of, in addition to SNPs, other types of DNA sequence alterations, such as insertions, deletions, duplications, inversions, etc. For the case of SNPs, additional genetic subtleties may be exploited. For example, SNPs have varying population allele frequencies. The minor allele frequency (MAF) is the frequency that the variant nucleotide occurs in a population, and MAFs can vary between ethnicities. A MAF of 0.01 for example occurs in the population at a frequency of 1%, whereas common SNPs can have MAFs as high as 0.5 or 50% of the population. Since submission of the parent application Ser. No. 15/916,052 human genomics research has advanced significantly with many more individual genomes across multiple populations sequenced and a greatly improved documentation of human genetic variation. The total number of identified human SNPs has increased from 39 million (39×10⁶) to approximately 335 million (335×10⁶). It is estimated that each individual has on average approximately 4-5 million SNPs. In addition to significantly more identified SNPs, those with substantially lower MAFs are observed. Deeper genomic sequencing has identified extremely rare SNPs that are present only once (termed “singletons”) in studies of five to ten thousand different individuals; it may be that some singletons are present only once in hundreds of thousands of individuals. This can be contrasted with current identity metrics; many people can have the same name and very many people do have the same date of birth. Thus, utilization of only a single SNP may provide a very effective proxy for identity verification. The differences between the MAFs of various SNPs may be judiciously incorporated into the IDV in various ways to improve robustness and security.

For example, consider the unlikely case that a F-IP had access to sophisticated genomic data and computational capabilities. If a particular SNP has a very low population allele frequency, F-IP could improve the probability of guessing or randomly selecting the correct DNA.seq of multiple IPs by using the high population frequency nucleotide for that SNP.

There are several corollaries to this possibility that could be utilized to improve IDV security. These include but are not limited to:
1. Use all or predominantly high MAF SNPs in the DNA.pos. This would make the probability of selecting/guessing the correct DNA.seq very close to random (3ⁿ for n SNPs). This would minimize the advantage to F-IP afforded by low frequency MAFs described above.
2. Utilize DNA.pos with all or predominantly SNPs for which IP has at least one allele of very low MAF. If F-IP does employ the strategy described above, this approach would decrease F-IP's probability of selecting/guessing the correct DNA.seq. It may also allow use of fewer total SNPs in DNA.pos.

Verification of identity for access to computers, servers, private networks or online (e.g. private databases or internet websites) has in some instances now progressed to the use of two factor authorization. One example is logging into a server using credentials such as username and password, and then being prompted by the server to provide a verification code. This typically involves, during the login process, requesting a verification code which is then sent by the server to the established personal address (e.g. e-mail address or text message) of the owner of the online account. Frequently it is a six digit number which is then entered and required to complete the login process. There are other types of verification codes, and sometimes multiple such credentials are required. These are collectively referred to as multifactor authorization (MFA).

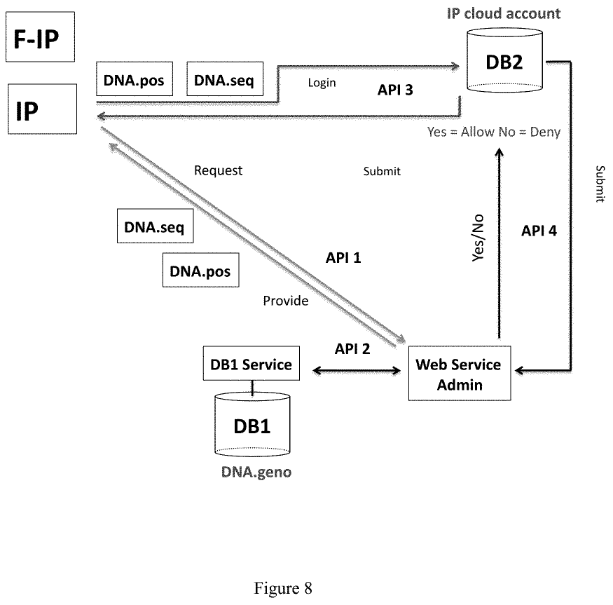

The IDV process of this invention may be performed entirely by and within a computer system. The computer architecture of one such embodiment appears in FIG. 8. In this case, an individual wishes to login to a personal online (or “cloud”) account (DB2). The owner of the online account (IP) has had their DNA.geno previously determined and deposited into a secure database (DB1). The DB1 Service interacts with the Admin Web Service through an application programming interface (API 2), but DB1 is not connected to the internet. IP and Admin communicate via API 1 with software on both the Admin Web Service and IPs computer or other electronic device. The IDV process is initiated when DB2 receives via API 3 a request, which provides pre-established credentials for an IP (e.g. username, password), to login to IPs personal online account. MFA is used in the login process as follows. DB2 replies to the login request via API 3 requesting provision of a DNA.pos file and a corresponding DNA.seq file. When the authentic IP initiates via API 3 the login and receives the MFA request, the IP sends a request via API 1 to Admin Web Service to provide a DNA.pos file and a corresponding DNA.seq file. Admin forwards this request via API 2 to the DB1 Service. The DB1 Service uses automated computational methods to extract from IPs DNA.geno in DB1 a DNA.pos and DNA.seq with desired properties and the DB1 Service forwards via API 2 the two files to Admin Web Service; these files are forwarded by Admin Web Service to IP via API 1. IP receives DNA.pos and DNA.seq via API 1 and IPs computer forwards via API 3 the two files to DB2. DB2 utilizes API 4 to forward those DNA.pos and DNA.seq files to Admin Web Service requesting verification that the credentials correspond to the IP in DB1. Admin forwards the two files to DB1 Service which then uses computational methods to extract from the IPs DNA.geno in DB1 the genotype of each DNA.pos and outputs it to a DNA.test file. DB1 Service uses computational methods to compare the submitted DNA.seq to the determined DNA.test. If there is perfect concordance of the two files, a confirmation of identity verification is transmitted to Admin Web Service; if there is discrepancy between DNA.seq and DNA.test a denial of identity verification is transmitted to Admin. Admin utilizes API 4 to send to DB2 a Yes (confirmation of identity verification) or a No (denial of identity verification) answer. For a Yes answer DB2 allows the login to DB2 to proceed, if DB2 receives a No answer the login request is rejected. It is possible that a fraudulent attempt (F-IP in FIG. 8) is made to login to IPs cloud account (using e.g. username, password), but without knowledge of or access to IPs genomic information the attempt would fail at the MFA step.

The parent application Ser. No. 15/916,052 noted, in addition to SNPs, DNA sequence variation of the type “insertions, deletions, inversions and/or repeats” (pgs. 7, 9, 12). These are collectively referred to as structural variants (SVs), and include variable number of tandem repeats (VNTRs), short tandem repeats (STRs) and large scale structural variation between individuals. The NCBI reference human genome assembly, which consists of a series of sequential Build numbers, was also discussed (the current is Build 38, abbreviated as GRCh38). In each reference genome assembly, SNPs occupy a specific position typically noted as chromosome number and nucleotide position (on either the positive or negative strand). The parent application taught (pg. 8) that the position number of a given SNP may change in different Build numbers due to inclusion or deletion of DNA sequences from previous builds, but that the genotype of the SNP remains unchanged. The necessity of preserving the genotype of each SNP requires that each reference genome build have a fixed number of total positions or coordinates (termed DNA.pos in the parent application) on each chromosome. As a consequence, in each region of each chromosome with SVs, a fixed number of VNTRs and STRs must be selected for the NCBI reference genome build. This means that any single human genome, with a different number of VNTRs or STRs, will have a different number of total coordinates on one or more of the chromosomes. Differences in SVs also distinguish an individual from others, and so are a measure of identity, but many SVs are not represented in NCBI reference human genome builds. A variety of methods have been developed and are available for alignment and annotation of STR data generated from NGS analysis.

Technology has also been developed for comparison of genomes that includes analysis of SVs. For example, the program Progressive Cactus (Armstrong, et al. Nature 587, 246-256; 12 Nov. 2020) enables the reference-free alignment of tens to thousands of large vertebrate genomes while maintaining high quality alignment. These analyses characterize, in addition to SNPs, structural variation between individual genomes. Recently, a method was developed that can efficiently map short read (NGS) sequencing reads to a collection of haplotypes threaded through a genome sequence (Siren et al. Science 374. eabg8871; 17 Dec. 2021). The method (“Giraffe”) maps next generation sequencing (NGS, or “short read”) data to a human pangenome at a speed comparable to that of standard methods mapping to a single reference genome. The term pangenome refers to, rather than a single reference genome, many complete individual genome assemblies. Siren et al. used Giraffe to genotype 167,000 SVs discovered in long-read studies of over 5,000 diverse human NGS genome sequences, and concluded that pangenomic approaches facilitate a more comprehensive characterization of genetic variation. The increased knowledge of human SVs obtained with these and other technologies will provide additional genetic variation data that can be used in the instant invention.

Haplotype is a set of DNA sequence variations that tend to be inherited together; this can refer to a combination of SNPs or other alleles. These regions are inherited together because they represent small lengths of DNA and recombination (crossovers between pairs of homologous chromosomes) are infrequent within the region. Some studies employ imputation to infer a specific SNP at a position on a haplotype based on an empirically determined SNP at a different position. Humans are diploid and inherit one set of chromosomes from each parent; haploid genotype refers to the set of alleles inherited from a single parent. With most DNA sequencing technologies, it has not been possible to deduce from which parent a given haplotype is derived without also having DNA sequence information from a child of the two parents. More recently (Ebert, et al. Science 372, eabf7117; 2 Apr. 2021), long-read and strand-specific sequencing technologies have been used on genomic DNA for which high quality full genome sequence was previously determined by next generation sequencing (NGS). From 32 diverse humans, 64 high quality assembled haplotypes were reported without sequence information of a child. Analyses of these haplotype-resolved human genomes revealed significantly more genetic variation, particularly with regard to structural variants. Whereas NGS typically identifies 5,000-10,000 SVs, long-read genome assemblies now routinely detect >20,000 SVs. These SVs, and those from other studies, represent additional sources of genetic variation that may be utilized in the IDV process of this invention. Additionally, the IDV process of the instant invention could be performed using haplotype-resolved individual human genomes.

An effort has been made to describe the invention and its practical applications in thorough detail. However, it should be understood that obvious variations will occur to those skilled in the fields to which this invention pertains, in light of this description, and that such variations are fully intended to fall within the purview of the invention even though not specifically referred to herein.

### Description of Specific Embodiments

The practice of the invention is further shown by reference to the following specific embodiments, which are included for illustrative purposes only and are not to be construed as limiting such practice to these working examples only. Abbreviations used in the examples are as defined above.

### Example 1

An IP (named 0001 in this example) requests an identity verification process (IDV) to confirm their identity for a transaction/purpose. 0001 had previously had DNA sequence analysis performed on her genome and the resulting DNA.geno file, which is named 000001_23_2012.08.18.0139.txt in this example, deposited in the DB in the directory DNA>GenExp_DNA.Seq. The DNA.geno file (000001_23_2012.08.18.0139.txt) is genotype data from a microarray that interrogated approximately 967,000 SNPs, and is included as part of this patent filing. In this example, a computer with the Unix operating system is used, and Unix commands (termed scripts) are used to automate the process. The information flow and actions in this example are depicted in FIGS. 1 and 2.

Step 1. 0001 initiates the IDV by submitting a request to the DB to provide two files based on her DNA.geno: DNA.pos and DNA.seq.
Step 2. In response to the 0001 request, DB generates two files:
DNA.pos

Six SNPs are randomly selected from the SNPs listed in 000001_23_2012.08.18.0139.txt. These six SNPs could be selected manually. Alternatively, the selection of SNPs could be automated by using any one of a large number of scripts that could be written by one of ordinary skill in the art, to generate a list of SNPs. The resulting list of SNPs extracted is designated DNA.pos. In this example the SNPs rs76255, rs4988235, rs2187668, rs6822844, rs6441961, and rs9851967 are present in DNA.pos.
DNA.seq

0001's genotype for the DNA.pos generated above is determined.

Using a computer running the Unix operating system, from the directory DNA>Computation_Dev the following script is run:

grep -Ew “rs762551|rs4988235|rs2187668|rs6822844|rs6441961|rs9851967”
../GenExp_DNA.Seq/000001_23_2012.08.18.0139.txt>

### ../GenExp_DNA.Seq/000001_23_2012.08.18.0139_IT_0002.txt

The computation generated the file 000001_23_2012.08.18.0139_IT_0002.txt, which appeared in the directory DNA>GenExp_DNA.Seq, and is designated herein DNA.seq. It provides the following information:

| | | | |
|---|---|---|---|
| rs4988235 | 2 | 136608646 | AA |
| rs6441961 | 3 | 46352384 | CT |
| rs9851967 | 3 | 188087628 | CT |
| rs6822844 | 4 | 123509421 | GT |
| rs2187668 | 6 | 32605884 | CC |
| rs762551 | 15 | 75041917 | AC |

The left column lists the specific rsID for each SNP queried in the script above, the second column lists the human chromosome on which said rsID is located, the third column lists the nucleotide position (in NCBI reference human genome assembly build 37, plus strand) on said chromosome where said rsID occurs, and the fourth column is IP 0001's genotype on the plus strand for said rsID.
Step 3. The DB provides DNA.pos and DNA.seq to 0001.
Step 4. 0001 provides the above two files (DNA.pos and DNA.seq) to an IDV to request verification of 0001 as the IP 0001.
Step 5. IDV submits the two files to DB
Step 6. DB extracts the DNA.pos genotypes from 0001's DNA.geno
Using a computer running the Unix operating system, from the directory DNA>Computation_Dev the following script is run:
   grep -Ew “rs762551|rs4988235|rs2187668|rs6822844|rs6441961|rs9851967”
   ../GenExp_DNA.Seq/000001_23_2012.08.18.0139.txt>

### ../GenExp_DNA.Seq/000001_23_2012.08.18.0139_ID_0002.txt

The computation generated the file 000001_23_2012.08.18.0139_ID_0002.txt in the directory GenExp_DNA.seq, which is designated herein DNA.test. It provides the following information:

| | | | |
|---|---|---|---|
| rs4988235 | 2 | 136608646 | AA |
| rs6441961 | 3 | 46352384 | CT |
| rs9851967 | 3 | 188087628 | CT |
| rs6822844 | 4 | 123509421 | GT |
| rs2187668 | 6 | 32605884 | CC |
| rs762551 | 15 | 75041917 | AC |

Step 7. The DNA.test is compared to DNA.seq submitted by IDV. This can be done manually. Alternatively, the comparison of SNP values in the two files could be automated by using a Unix command that could be written by one of ordinary skill in the art.
Step 8. In this example, there is a perfect match between DNA.test and DNA.seq. Every SNP included in DNA.pos appears in DNA.geno. The chromosome number and nucleotide position on that chromosome of each SNP in DNA.pos and DNA.seq are identical. Most importantly, the genotype of each SNP in DNA.pos and DNA.seq are identical.
Step 9. DB instructs IDV to confirm the identity of 0001
Step 10. IDV confirms the identity of 0001, and the transaction/purpose for which IDV was sought can be authorized to proceed.

### Example 2

A separate entity (named 000X in this example) wishes to be identified as an IP (named 0001 in this example), most likely for nefarious purposes, but in any event the IDV is not authorized by 0001. The information flow and actions in this example are depicted in FIG. 2. This is a fraudulent attempt to obtain identity verification as 0001, and 000X is indicated as F-IP in FIG. 2.

0001 had previously had DNA sequence analysis performed on her genome and the resulting DNA.geno file deposited in the DB in the directory DNA>GenExp_DNA.Seq. In this example, the DNA.geno of IP 0001 is named 000001_23_2012.08.18.0139.txt, the genotype data is from a microarray that interrogated approximately 967,000 SNPs, and the data file (000001_23_2012.08.18.0139.txt) is included in this patent filing. In this example, a computer with the Unix operating system is used, and Unix commands (termed scripts) are used to automate the process.

Step 1. 000X initiates the IDV by submitting two files to IDV: DNA.pos and DNA.seq. In this example the SNPs present in DNA.pos are rs76255, rs4988235, rs2187668, rs6822844, rs6441961, and rs9851967.

Without access to 0001's DNA.geno, 000X will need to submit a single genotype from the 729 possible genotypes for 6 SNPs (3⁶). In this example 000X submits the following DNA.seq:

| | | | |
|---|---|---|---|
| rs4988235 | 2 | 136608646 | AA |
| rs6441961 | 3 | 46352384 | TT |
| rs9851967 | 3 | 188087628 | CC |
| rs6822844 | 4 | 123509421 | GG |
| rs2187668 | 6 | 32605884 | CC |
| rs762551 | 15 | 75041917 | AA |

Step 2. IDV submits the two files (DNA.pos and DNA.seq) to DB
Step 3. DB extracts the rsID genotypes from 0001's DNA.geno, using the DNA.pos submitted by 000X.
Using a computer running the Unix operating system, from the directory DNA>Computation_Dev the following script is run:
   grep -Ew “rs762551|rs4988235|rs2187668|rs6822844|rs6441961|rs9851967”
   ../GenExp_DNA.Seq/000001_23_2012.08.18.0139.txt>

### ../GenExp_DNA.Seq/000001_23_2012.08.18.0139_ID_0002.txt

The computation generated the file 000001_23_2012.08.18.0139_ID_0002.txt in the directory GenExp_DNA.seq, which is designated herein DNA.test. It provides the following information:

| | | | |
|---|---|---|---|
| rs4988235 | 2 | 136608646 | AA |
| rs6441961 | 3 | 46352384 | CT |
| rs9851967 | 3 | 188087628 | CT |
| rs6822844 | 4 | 123509421 | GT |
| rs2187668 | 6 | 32605884 | CC |
| rs762551 | 15 | 75041917 | AC |

4. The DNA.test so obtained is compared to DNA.seq submitted to DB by IDV. This can be done manually. Alternatively, the comparison of SNP values in the two files could be automated by using a Unix based script that could be written by one of ordinary skill in the art.
5. In this example, there are differences between DNA.test and DNA.seq. Specifically, the genotypes of rs6441961, rs9851967, rs6822844 and rs762551 in DNA.test determined on the DNA.geno of IP (0001 in this example) is different than the DNA.seq submitted to IDV.
6. DB instructs IDV to deny verification of 000X (F-IP) as 0001 (IP).

### Example 3

A separate entity (named 000Y in this example) wishes to be identified as an IP (named 0001 in this example), most likely for nefarious purposes, but in any event the IDV is not authorized by 0001. The information flow and actions in this example are depicted in FIG. 2. This is a fraudulent attempt to obtain identity verification as 0001, and 000Y is indicated as F-IP in FIG. 2.

0001 had previously had DNA sequence analysis performed on her genome and the resulting DNA.geno file deposited in the DB in the directory DNA>GenExp_DNA.Seq. In this example, the DNA.geno of IP 0001 is named 000001_23_2012.08.18.0139.txt, the genotype data is from a microarray that interrogated approximately 967,000 SNPs, and the data file (000001_23_2012.08.18.0139.txt) is included in this patent filing. In this example, a computer with the Unix operating system is used, and Unix commands (termed scripts) are used to automate the process.

Step 1. 000Y initiates the IDV by submitting two files to IDV: DNA.pos and DNA.seq. In this example the SNPs present in DNA.pos are rs76255, rs4988235, rs2187668, rs6822844, rs6441961, rs9851967, and rs1799752.
Step 2. IDV submits the two files (DNA.pos and DNA.seq) to DB.
Step 3. DB tests 0001's DNA.geno for the presence of the 7 SNPs. This can be performed manually by viewing the 000001_23_2012.08.18.0139.txt file in a text editor and using the command “find” for each SNP. Alternatively, a Unix command could be written by one of ordinary skill in the art to determine the presence or absence of each SNP in DNA.geno. The analysis demonstrates that the first 6 SNPs above appear in 0001's DNA.geno. However rs1799752 is not included in the file, presumably because rs1799752 was not interrogated in the microarray used to determine 0001's DNA.geno.

Based on this discrepancy alone, DB can instruct IDV to deny verification of 000Y as 0001.

Step 4. The information submitted by 000Y can be further analyzed.

Without access to 0001's DNA.geno, 000Y will need to submit a single genotype from the 2,187 possible genotypes for 7 SNPs (3⁷). In this example 000Y submits the following DNA.seq:

| | | | |
|---|---|---|---|
| rs4988235 | 2 | 136608646 | AA |
| rs6441961 | 3 | 46352384 | TT |
| rs9851967 | 3 | 188087628 | CC |
| rs6822844 | 4 | 123509421 | GG |
| rs2187668 | 6 | 32605884 | CC |
| rs762551 | 15 | 75041917 | AA |
| rs1799752 | 17 | | deletion/deletion |

DB extracts the rsID genotypes from 0001's DNA.geno, using the DNA.pos submitted by 000Y. Using a computer running the Unix operating system, from the directory DNA>Computation_Dev the following script is run:

grep -Ew “rs762551|rs4988235|rs2187668|rs6822844|rs6441961|rs9851967|rs1799752”
../GenExp_DNA.Seq/000001_23_2012.08.18.0139.txt>

### ../GenExp_DNA.Seq/000001_23_2012.08.18.0139_ID_0002.txt

The computation generated the file 000001_23_2012.08.18.0139_ID_0002.txt in the directory GenExp_DNA.seq, which is designated herein DNA.test. It provides the following information:

| | | | |
|---|---|---|---|
| rs4988235 | 2 | 136608646 | AA |
| rs6441961 | 3 | 46352384 | CT |
| rs9851967 | 3 | 188087628 | CT. |
| rs6822844 | 4 | 123509421 | GT |
| rs2187668 | 6 | 32605884 | CC |
| rs762551 | 15 | 75041917 | AC |

5. The DNA.test so obtained is compared to DNA.seq submitted to DB by IDV. This can be done manually. Alternatively, the comparison of SNP values in the two files could be automated by using a Unix based script that could be written by one of ordinary skill in the art.
6. In this example, there are a number of differences between DNA.test and DNA.seq. First, the result for rs1799752 does not appear in DNA.test for the reason explained in Step 3 above. The IDV request could be denied based on this discrepancy alone. Furthermore, the genotypes of rs6441961, rs9851967, rs6822844 and rs762551 in DNA.test determined on the DNA.geno of IP (0001 in this example) are different than those submitted by 000Y in DNA.seq.
7. DB instructs IDV to deny verification of 000Y as 0001.

1. A method of personal identity verification, which method is performed using a computer system that comprises a plurality of networked computers to perform computational analysis of genomic DNA, the method comprising:
(a) providing a computerized database comprising identity information for a plurality of individuals, which identity information comprises
(1) one or more static identifier selected from a group including name, date of birth, social security number, drivers license number, mother maiden name, fingerprint, eyescan, retina scan, a number, or a combination of numbers and letters, and
(2) a first directory of genomic DNA sequence information for each of the plurality of individuals in the computerized database, wherein the DNA sequence information for any individual having genomic DNA sequence information stored in the computerized database is used to confirm or deny a request for identity verification as that individual and comprises a plurality of reference genome positions that include one or more positions of DNA sequence variation of the type single nucleotide polymorphism, inversion or structural variant, and a genotype for the given individual at each reference genome position represented in the DNA sequence information for the given individual, and
(3) a second directory of DNA position files, each DNA position file comprising reference genome positions in the genomic sequence information of an individual in the first directory, that have been provided by the computer system to an individual for use in personal identity verification, and to which directory additional DNA position files may be deposited;
(b) the computer system receiving from a requestor a request to be verified as a given individual, which request comprises static identifier data;
(c) provided that the static identifier data of step (b) corresponds to static identifier data of an individual represented in the computerized database, using the computer system to generate and send to the requestor a response, wherein the response comprises requesting provision of a DNA position file and provision of a corresponding DNA sequence file that comprises the genotype of each reference genome position in the DNA position file;
(d) using the computer system, the requestor replies to the response of step (c) by electronically submitting a DNA position file and a DNA sequence file to the computer system;
(e) using the computer system to generate a DNA test file by extracting from the given individual's genomic sequence information in the first directory the genotypes that correspond to the reference genome positions provided in the DNA position file of step (d);
(f) using the computer system to compare the genotypes of the reference genome positions in the DNA sequence file of step (d) with those of the DNA test file of step (e); and
(g) using the computer system to either
(1) deny the identity verification request of step (b) if there is any mismatch between the genotypes of the DNA test file and the DNA sequence file at step (f), or
(2) confirm the identity verification request of step (b) if there is perfect concordance of the genotypes of the DNA test file and the DNA sequence file at step (f).

2. The method of claim 1 wherein the given individual's genomic sequence information in the first directory is
a DNA nucleotide sequence of the entire genome of the given individual, or
a DNA nucleotide sequence subset of the entire genome of the given individual, or
a combination, either overlapping or non-overlapping, of DNA nucleotide sequence subsets of the entire genome of the given individual, or
non-contiguous single nucleotides from the entire genome of the given individual, or
any combination of the above.

3. (canceled)

4. The method of claim 1 wherein the computer system is used to determine whether all reference genome positions in the DNA position file of step (d) are present in the genomic DNA sequence information for the given individual in the first directory, and either
(1) proceeding to step (e) if all reference positions are present, or
(2) deny the identity verification request of step (b) if a sequence variant is not present and terminate the identity verification process.

5. The method of claim 1 wherein the computer system is used to analyze whether the DNA position file submitted at step (d) includes at least one position of DNA sequence variation of the type single nucleotide variation, inversion or structural variant and either
(a) proceed to step (e) if a sequence variant is present, or
(b) deny the identity verification request of step (b) if a sequence variant is not present and terminate the identity verification process

6. The method of claim 1, wherein
(a) the computer system receives from a given individual an electronic request to initiate an identity verification process, which given individual is one of the plurality of individuals having identity information stored in the computerized database, and
(b) the computer system generates a command to extract from the given individual's genomic sequence information in the first directory a DNA position file and a corresponding DNA sequence file and electronically transmits the DNA position file and the corresponding DNA sequence file to the given individual.

7. The method of claim 6 wherein
the DNA position file of step (b) is a subset of the reference genome positions in the genomic sequence information of the given individual in the first directory and claim 6 step (b) comprises:
(1) using the computer system to initially access all DNA position files of the given individual that are stored in the second directory and then select one or more reference genome positions from the given individual's genomic sequence information in the first directory that are not present in the DNA position files of the given individual in the second directory;
(2) using the computer system to generate a DNA position file, that includes the reference genome positions selected in step (1), and a corresponding DNA sequence file and then electronically transmitting the DNA position file and the corresponding DNA sequence file to the given individual; and
(3) using the computer system to associate the DNA position file of step (2) with the given individual and depositing it in the second directory.

8. The method of claim 1 wherein the computer system determines whether the DNA position file of step (d) includes reference genome positions that are not present in reference genome positions for the given individual in the second directory, and either
(1) proceed to step (e) if one or more new reference genome positions are present, or
(2) deny the identity verification request of step (b) if new reference genome positions are not present and terminate the identity verification process.

9. The method of claim 1, wherein
(a) the genomic DNA sequence information of an individual that is stored in the first directory is a subset of the entire genomic sequence information of the given individual, and is removed from the database and not used for subsequent identity verification requests, and
(b) new genomic DNA sequence information of the given individual that comprises a plurality of reference genome positions that are either non-overlapping or partially overlapping with the genomic sequence information removed in step (a), and includes DNA sequence variation that is not present in the genomic sequence information removed in step (a), is deposited in the first directory.

10. The method of claim 6, wherein
(a) the DNA sequence file provided to the given individual at step (b) is retained as a digital signature in the computerized database and also transmitted to the given individual for future use as a digital signature;
(b) the digital signature is updated in the computerized database and provided to the given individual each time a new DNA position file and a corresponding DNA sequence file is transmitted to the given individual;
(c) the response of claim 1 step (c) also requests provision of a file comprising a digital signature;
(d) the requestor submits to the computer system at claim 1 step (d) a digital signature in addition to a DNA position file and a DNA sequence file;
(e) the computer system compares the digital signature of step (d) with the most recent digital signature for the given individual stored in the computerized database and either
(1) proceeds to step (e) if the digital signature is the same as the previous digital signature for the given individual stored in the computerized database, or
(2) deny the identity verification request of step (b) if the digital signature is different and terminate the identity verification process.

11. The method of claim 1 wherein the identity verification request of step (b) is used for multifactor authorization to login to a computer, server, network or internet account, and either
claim 1 step (g)(1) results in denial of login to the computer, server network, or internet account, or
claim 1 step (g)(2) results in login to the computer, server, network or internet account.

12. The method of claim 1 wherein the first directory is maintained using blockchain technology

13. The method of claim 12 wherein the blockchain is either a public blockchain, a private blockchain or a consortium controlled blockchain.

14. The method of claim 12 wherein the blockchain employs a consensus protocol selected from a group including proof of work, proof of stake, proof of capacity/proof of space, delegated proof of stake, proof of authority, practical byzantine fault tolerance, or proof of elapsed time.

15. A method of multifactor authorization, which method is performed using a computer system that comprises a plurality of physically distinct networked computers that interact through the internet and a non-internet private network, the method comprising:
(a) providing a non-internet private database comprising identity information for a plurality of individuals, which identity information comprises
(1) one or more static identifier selected from a group including name, date of birth, social security number, drivers license number, mother maiden name, fingerprint, eyescan, retina scan, a number, a combination of numbers and letters, or a username and password, and
(2) a directory of genomic DNA sequence information for each of the plurality of individuals in the computerized database, wherein the DNA sequence information for any individual having genomic DNA sequence information stored in the computerized database is used for multifactor authorization as that individual and comprises a plurality of reference genome positions that include one or more DNA sequence variations of the type single nucleotide polymorphism, inversion or structural variant, and a genotype for the given individual at each reference genome position represented in the DNA sequence information for the given individual;
(b) an individual person using a first application programming interface requests login to a personal online account on a remote server by submitting static identifier;
(c) provided that the static identifier of step (b) corresponds to an account on the remote server, the remote server replies via the first application programming interface requesting provision of DNA credentials;
(d) the individual person communicates via a second application programming interface with a web service to provide static identifier and request DNA credentials;
(e) provided that the static identifier of step (d) corresponds to one of the plurality of individuals in the non-internet private database, the webservice communicates via a third application programming interface with a database service of the non-internet private database and obtains from the non-internet private database a DNA position file comprising reference genome positions in the genomic sequence information of the individual person associated with the static identifier of step (d) and a corresponding DNA sequence file that comprises the genotype of each reference genome position in the DNA position file;
(f) the webservice provides via the second application programming interface the DNA position and the DNA sequence files to the individual person;
(g) the individual person transmits via the first application programming interface the DNA position and DNA sequence files to the remote server;
(h) the remote server receives the DNA position and DNA sequence files and transmits the files via a fourth application programming interface to the web service, requesting verification that the DNA position and the DNA sequence files correspond to genomic DNA information of the person associated with the static identifier of step (b);
(i) the webservice via the third application programming interface uses the database service of the secure private server to generate a DNA test file by extracting from the genomic DNA sequence information associated with the static identifier of step (b) the genotypes that correspond to the reference genome positions provided in the DNA position file of step (h);
(j) the web service uses the database service of the secure private server to compare the DNA test file of step (i) with the DNA sequence file received at step (h) and either
(1) generates a no if there is any mismatch between the genotypes of the DNA test file and the DNA sequence file, or
(2) generates a yes if there is perfect concordance of the genotypes of the DNA test file and the DNA sequence file;
(k) the web service transmits via the fourth application programming interface the result of step (j) to the remote server; and
(l) the remote server either
(1) denies the login request of step (b) if a no is received at step (k) and prevents access to the personal online account, or
(2) allows the login request of step (b) if a yes is received at step (k) and permits access to the personal online account.
